(19) United States
(12) Patent Application Publication
(10) Pub. No.: US 2023/0401712 A1
(43) Pub. Date: Dec. 14, 2023

(54) METHOD FOR DETERMINING CARDIAC CORONARY ARTERY IMAGING PHASE, ELECTRONIC DEVICE, AND STORAGE MEDIUM

(71) Applicant: SHANGHAI UNITED IMAGING HEALTHCARE CO., LTD., Shanghai (CN)

(72) Inventor: YI WANG, Shanghai (CN)

(21) Appl. No.: 18/207,760

(22) Filed: Jun. 9, 2023

(30) Foreign Application Priority Data
Jun. 9, 2022 (CN) 202210647665.6

(51) Int. Cl. G06T 7/00; G06T 7/136; G06T 7/11; G06T 7/174; G06T 7/73; G06T 11/00

(52) U.S. Cl. CPC G06T 7/0014; G06T 7/136; G06T 7/11; G06T 7/174; G06T 7/74; G06T 11/006; G06T 2207/30168; G06T 2207/30101; G06T 2207/30048; G06T 2207/20021; G06T 2207/20036; G06T 2207/10081; G06T 2210/41; G06T 2211/40

(57) ABSTRACT

The present disclosure relates to a method for determining a cardiac coronary artery imaging phase. The method includes acquiring a plurality of phase images of a cardiac coronary artery, extracting one or more target coronary arteries based on the plurality of phase images respectively to obtain a plurality of corresponding to-be-evaluated images, calculating image quality scores of the one or more target coronary arteries in each of the to-be-evaluated images, performing a weighted calculation according to the image quality score and weighted parameters of the one or more target coronary arteries to obtain a quality score of each of the to-be-evaluated images, and determining a required imaging phase of the cardiac coronary artery based on the quality scores of the plurality of to-be-evaluated images.

### CROSS-REFERENCE TO RELATED APPLICATIONS

This application claims priority to Chinese Patent Application No. 202210647665.6, entitled “METHOD FOR DETERMINING CARDIAC BLOOD VESSEL IMAGING PHASE AND APPARATUS, ELECTRONIC DEVICE, AND STORAGE MEDIUM” filed Jun. 9, 2022, the content of which is hereby incorporated by reference in its entirety.

### TECHNICAL FIELD

The present disclosure relates to the field of medical imaging technologies, particularly to a method for determining a cardiac coronary artery imaging phase, an electronic device, and a storage medium.

### BACKGROUND

During medical image reconstruction, multi-phase data may be acquired by using a Computed Tomography (CT) scanner, and image reconstruction is performed according to the multi-phase data to obtain a plurality of images of diseased parts. Automatic determination of suitable phase points for image reconstruction can improve the quality of a target reconstructed image.

In cardiac scanning, the quality of coronary arteries in a coronary artery determines the quality of a cardiac image. Due to cardiac motion during CT scanning, there is a need to select an optimal imaging phase of a cardiac coronary artery for the image reconstruction during image reconstruction. However, motion patterns of different coronary arteries are different. From clinical experience, generally, the optimal image quality of different coronary arteries (such as a left main artery, a left anterior descending artery, a left circumflex artery, and a right coronary artery) may be in different time phases. In this case, a phase obtained by using an optimal phase algorithm of a global image may not be an optimal phase of a target coronary artery, which affects the imaging quality of the target coronary artery.

### SUMMARY

Embodiments of the present disclosure provide a method for determining a cardiac coronary artery imaging phase, an electronic device, and a storage medium.

One aspect of the present disclosure provides a method for determining a cardiac coronary artery imaging phase which includes acquiring a plurality of phase images of a cardiac coronary artery, extracting one or more target coronary arteries based on the plurality of phase images respectively to obtain a plurality of corresponding to-be-evaluated images, calculating image quality scores of the one or more target coronary arteries in each of the to-be-evaluated images, performing weighted calculation according to the image quality scores of the one or more target coronary arteries and the weighted parameters of the one or more target coronary arteries to obtain a quality score of each of the to-be-evaluated images, and determining a required imaging phase of the cardiac coronary artery based on the quality scores of the plurality of to-be-evaluated images.

In some embodiments, extracting one or more target coronary arteries based on the plurality of phase images respectively to obtain the plurality of corresponding to-be-evaluated images includes performing target coronary artery positioning on each of the phase images, determining an image segmentation threshold, and extracting the one or more target coronary arteries from the phase image after the target coronary artery positioning according to the image segmentation threshold to obtain the corresponding to-be-evaluated image.

In some embodiments, determining the image segmentation threshold includes acquiring a pixel value or CT value of the phase image, acquiring a preset subdivision parameter corresponding to the target coronary artery, and calculating the image segmentation threshold of the phase image according to the pixel value or CT value and the subdivision parameter.

In some embodiments, prior to extracting the one or more target coronary arteries from the phase image after the target coronary artery positioning according to the image segmentation threshold to obtain the corresponding to-be-evaluated image, the method further includes marking a segmentation center according to a position of the target coronary artery in the phase image, and delimiting a segmentation region based on the segmentation center, so as to segment the phase image based on the segmentation region.

In some embodiments, prior to extracting the one or more target coronary arteries from the phase image after the target coronary artery positioning according to the image segmentation threshold to obtain the corresponding to-be-evaluated image, the method further includes reconstructing the phase image by an image interpolation operation.

In some embodiments, prior to extracting the one or more target coronary arteries from the phase image after the target coronary artery positioning according to the image segmentation threshold to obtain the corresponding to-be-evaluated image, the method further includes performing a morphological operation on the phase image to weaken an image background.

In some embodiments, calculating image quality scores of the one or more target coronary arteries in each of the to-be-evaluated images includes calculating a plurality of quality evaluation indexes corresponding to the one or more target coronary arteries in each of the to-be-evaluated images, determining weighting parameters of the plurality of quality evaluation indexes, and performing a weighted calculation based on the plurality of quality evaluation indexes corresponding to the one or more target coronary arteries and the weighting parameters of the quality evaluation indexes, to obtain the quality scores of the one or more target coronary arteries in the to-be-evaluated image.

In some embodiments, determining the required imaging phase of the cardiac coronary artery based on the quality scores of the plurality of to-be-evaluated images includes acquiring the plurality of to-be-evaluated images within a preset single phase sliding window including multiple time phases, and performing filtering to obtain common slices and extra slices that correspond to each time phase, calculating, based on image quality scores of the common slices and the extra slices in a single time phase and a number of image layers of the single time phase, an image quality score of the corresponding time phase, and repeating the above step to calculate the quality scores of the plurality of to-be-evaluated images in each time phase within the single phase sliding window, and determining the required imaging phase of the cardiac coronary artery according to the quality scores the plurality of to-be-evaluated images.

In some embodiments, the calculating, based on the image quality scores of the common slices and the extra slices in the single time phase and the number of image layers of the single time phase, the image quality score of the corresponding time phase includes calculating, based on quality scores of the common slices and a number of layers of each time phase with the common slices in the single time phase within the single phase sliding window, an average score of the corresponding time phase, calculating an inter-phase offset score according to a number of layers of the common slices in the single time phase and a mean value of a number of layers of each time phase, determining an intra-phase offset score based on quality scores of the extra slices in the single phase, and performing a weighted calculation according to the average score, the inter-phase offset score, and the intra-phase offset score to obtain the image quality score of the corresponding time phase.

In some embodiments, the method further includes performing image registration in the z-direction on the at least one target coronary artery of the to-be-evaluated image in each time phase to determine a position of the at least one target coronary artery in the z-direction corresponding to each time phase; selecting the to-be-evaluated images within a preset similarity range based the positions of the at least one target coronary artery after image registration; and determining the required imaging phase of the cardiac coronary artery based on quality scores of the selected to-be-evaluated images.

Another aspect of the present disclosure provides an electronic device which includes a memory and a processor. The memory stores a computer program, and the processor is configured to execute the computer program to perform a method for determining a cardiac coronary artery imaging phase which includes acquiring a plurality of phase images of a cardiac coronary artery, extracting one or more target coronary arteries based on the plurality of phase images respectively to obtain a plurality of corresponding to-be-evaluated images, calculating image quality scores of the one or more target coronary arteries in each of the to-be-evaluated images, performing a weighted calculation according to the image quality scores of the one or more target coronary arteries and the weighted parameters of the one or more target coronary arteries to obtain a quality score of each of the to-be-evaluated images, and determining a required imaging phase of the cardiac coronary artery based on the quality scores of the plurality of to-be-evaluated images.

Yet another aspect of the present disclosure provides a computer-readable storage medium storing a computer program. The computer program, when executed by a processor, causes the processor to perform a method for determining a cardiac coronary artery imaging phase which includes acquiring a plurality of phase images of a cardiac coronary artery, extracting one or more target coronary arteries based on the plurality of phase images respectively to obtain a plurality of corresponding to-be-evaluated images, calculating image quality scores of the one or more target coronary arteries in each of the to-be-evaluated images, performing a weighted calculation according to the image quality scores of the one or more target coronary arteries and the weighted parameters of the one or more target coronary arteries to obtain a quality score of each of the to-be-evaluated images, and determining a required imaging phase of the cardiac coronary artery based on the quality scores of the plurality of to-be-evaluated images.

Details of one or more embodiments of the present disclosure are set forth in the following accompanying drawings and descriptions, so that other features, objectives, and advantages of the present disclosure become more obvious.

### BRIEF DESCRIPTION OF THE DRAWINGS

The accompanying drawings described herein are used to provide a further understanding of the present disclosure, and form part of the present disclosure. Exemplary embodiments of the present disclosure and descriptions thereof are used to explain the present disclosure, and do not constitute any inappropriate limitation on the present disclosure. In the accompanying drawings,

FIG. 1 is a schematic flowchart of a method for determining a cardiac coronary artery imaging phase according to an embodiment of the present disclosure;

FIG. 2 is a schematic flowchart of extracting one or more target coronary arteries based on a plurality of phase images respectively to obtain a plurality of corresponding to-be-evaluated images according to an embodiment of the present disclosure;

FIG. 3 is a schematic diagram of a result of target coronary artery positioning on phase images according to an embodiment of the present disclosure;

FIG. 4 is a schematic flowchart of calculating image quality scores corresponding to one or more target coronary arteries in a single to-be-evaluated image according to an embodiment of the present disclosure;

FIG. 5 is a schematic flowchart of a method for determining a cardiac coronary artery imaging phase according to another embodiment of the present disclosure;

FIG. 6 is a structural block diagram of a cardiac coronary artery imaging phase determination apparatus according to an embodiment of the present disclosure; and

FIG. 7 is a schematic structural diagram of an electronic device according to an embodiment of the present disclosure.

### DETAILED DESCRIPTION

To make the objectives, technical solutions, and advantages of the present disclosure clearer, the present disclosure is described and illustrated below with reference to the accompanying drawings and embodiments. It should be understood that specific embodiments described herein are merely used to explain the present disclosure but are not intended to limit the present disclosure. All other embodiments obtained by those of ordinary skill in the art based on the embodiments provided in the present disclosure without creative efforts shall fall within the protection scope of the present disclosure.

Obviously, the accompanying drawings described below are only some examples or embodiments of the present disclosure. Those of ordinary skill in the art may apply the present disclosure to other similar scenarios according to these accompanying drawings without creative efforts. In addition, it should be further understood that, although efforts made in this development process may be complicated and tedious, for those of ordinary skill in the art related to the content disclosed in the present disclosure, some designs, manufacturing or production changes based on the technical content disclosed in the present disclosure are merely conventional technical means, and should not be understood as insufficient content disclosed in the present disclosure.

The “embodiments” as referred to in the present disclosure means that particular features, structures or characteristics described with reference to the embodiments may be included in at least one embodiment of the present disclosure. Phrases appearing at various positions of the specification neither always refer to the same embodiment, nor separate or alternative embodiments that are mutually exclusive with other embodiments. It is explicitly and implicitly understood by those of ordinary skill in the art that the embodiments described in the present disclosure may be combined with other embodiments without conflict.

Unless defined otherwise, technical and scientific terms as referred to in the present disclosure have the same meanings as would generally be understood by those skilled in the technical field of the present disclosure. Similar words such as “a”, “an”, “one”, and “the” as referred to in the present disclosure do not indicate a limitation on a quantity, and may indicate singular or plural forms. Terms such as “comprise”, “include”, “have”, and other variants thereof as referred to in the present disclosure are intended to cover a non-exclusive inclusion, for example, processes, methods, systems, products or devices including a series of steps or units are not limited to these steps or units listed explicitly, but rather include other steps or units not listed, or other steps or units inherent to these processes, methods, systems, products or devices. Similar words such as “connect”, “join”, and “couple” as referred to in the present disclosure are not limited to physical or mechanical connections, but may include electrical connections, whether direct or indirect. “A plurality of” as referred to in the present disclosure means two or more. “And/or” indicates an association relationship describing associated objects, indicating that three relationships may exist. For example, “A and/or B” indicates that there are three cases of A alone, A and B together, and B alone. The character “I” generally means that the associated objects are in an “or” relationship. The terms “first”, “second”, “third”, and the like as referred to in the present disclosure are only intended to distinguish similar objects, and do not represent a specific ranking of objects.

A CT device uses X-rays to scan a certain thickness from many directions of a human body. A detector converts attenuated X-rays into visible light, and then converts the visible light into an electric signal. Finally, after the analog to digital conversion of the electric signal, a computer device performs image reconstruction to obtain a final CT image. When CT is used for cardiac image reconstruction, the clarity of coronary angiography is the key to determining the quality of a cardiac reconstruction image. Due to the physiological characteristics of a heart, there is a need to select optimal phase data for image reconstruction during the image reconstruction.

With vigorous development of computer technologies, acquisition, processing, display, and storage of medical images have been digitized, and image data processed by doctors and the workload of film reading have increased exponentially. Controlling human factors and improving the quality of image acquisition and processing are keys to correct disease diagnosis.

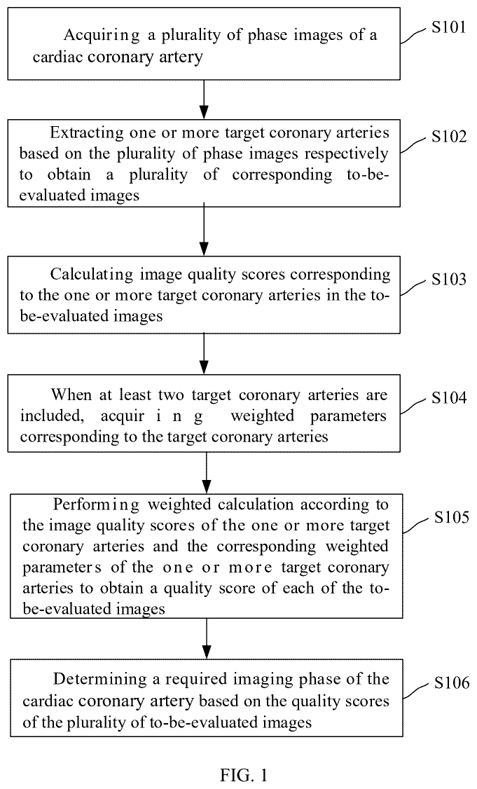

This embodiment further provides a method for determining a cardiac coronary artery imaging phase. FIG. 1 is a schematic flowchart of a method for determining a cardiac coronary artery imaging phase according to an embodiment of the present disclosure. As shown in FIG. 1, the process includes the following steps.

In step S101, a plurality of phase images of a cardiac coronary artery are acquired.

In this embodiment, during normal scanning of the CT, a scanned cardiac coronary artery is continuously scanned for a period of time, and corresponding scanning data is obtained. A plurality of to-be-evaluated images are acquired according to the scanning data.

Specifically, multi-phase reconstruction may be performed according to preset reconstruction parameters. The reconstruction parameters include a preset reconstruction center and a preset reconstruction range, and cardiac protocol common parameters may be used. A position of a coronary artery in a chest cavity is not fixed, and the coronary artery has a curved and changing shape. Therefore, a reconstruction range of a multi-phase image is required to cover an entire to-be-scanned region, that is, all phases that can be reconstructed. At the same time, in consideration of a balance between image resolution and computational efficiency, a reconstruction matrix and a reconstruction field of view should not be excessively small or excessively large.

In step S102, one or more target coronary arteries are extracted based on the plurality of phase images respectively to obtain a plurality of corresponding to-be-evaluated images.

In this embodiment, the target coronary artery may be extracted from the plurality of phase images by using a neural network model based on deep learning. Details are not described herein in the present disclosure. Optionally, when there are a plurality of target coronary arteries, each to-be-evaluated image includes a plurality of extracted target coronary arteries. Specifically, a deep learning neural network structure, training feature parameters, and loss functions are optimized, and different extraction scales may be determined. For example, only a left coronary artery and a right coronary artery are extracted, or more subdivided 16-level coronary arteries are extracted. After quality score evaluation on the to-be-evaluated image, quality scores of different target coronary arteries can be obtained, so as to select an optimal imaging phase for imaging.

Certainly, in other embodiments, the target coronary artery may alternatively be extracted based on a method such as image processing, enhanced filtering, or region growing, which is not limited herein in the present disclosure.

In step S103, image quality scores corresponding to the one or more target coronary arteries in each of the to-be-evaluated images are calculated.

Specifically, in this embodiment, the quality of the target coronary artery may be evaluated by using any quality evaluation index such as inter-regional contrast, intra-regional uniformity, shape smoothness measure, and a regional shape area difference, or a plurality of evaluation indexes are combined to make an evaluation result more comprehensive.

In step S104, when at least two target coronary arteries are included, weighted parameters corresponding to the target coronary arteries are acquired.

In this embodiment, the target coronary artery may be at least two of large branches such as a left coronary artery and a right coronary artery, or small branches such as a left anterior descending artery and a left circumflex artery, which is not limited in the present disclosure. When at least two target coronary arteries are included, the weighted parameters for the target coronary arteries can be preset, or customized by the user as required. Exemplarily, a degree of importance of the qualities of different coronary arteries in the quality of an entire cardiovascular image may be judged according to a clinical requirement, and the weighted parameters of the different target coronary arteries may be configured accordingly.

In step S105, a weighted calculation is performed according to the image quality scores and the corresponding weighted parameters of the target coronary arteries to obtain a quality score of each of the to-be-evaluated images.

Specifically, after the weighted parameters corresponding to the target coronary arteries are acquired, the image quality score and the corresponding weighted parameter of each of the target coronary arteries are weighted and summed to obtain the quality score of each of the to-be-evaluated images. Ranking the phases corresponding to the global coronary image qualities according to the quality scores of the to-be-evaluated images reflects the actual clinical needs of the user, and the user can determine an optimal phase for reconstruction according to a use purpose, thereby improving imaging quality of the target coronary artery.

Exemplarily, when image quality scores of a plurality of target coronary arteries in a certain to-be-evaluated image are X₁, X₂, and X₃ respectively and the corresponding weighted parameters are w₁, w₂, and w₃ respectively, it may be determined that the quality score of the to-be-evaluated image is Y=X₁*w₁+X₂*w₂+X₃*w₃.

It is to be noted that when there is only one target coronary artery, the only weighted parameter is 100%.

In step S106, a required imaging phase of the cardiac coronary artery is determined based on the quality scores of the plurality of to-be-evaluated images.

In this embodiment, “a required imaging phase of the cardiac coronary artery is determined based on the quality scores of the plurality of to-be-evaluated images” means that the user may select phases at different ranking positions for reconstruction according to a use purpose of the phase image.

Specifically, quality score ranking results may be determined based on the quality scores of the plurality of to-be-evaluated images, an index corresponding to one ranking result represents a ranking of coronary artery quality under this phase, and an optimal phase is a phase ranked as 1. The user may select a phase for reconstruction according to his/her own requirement. Specifically, (1) the optimal phase may be selected for reconstruction for coronary diagnosis. (2) The optimal phase may be selected for motion correction and a non-optimal phase (worst or intermediate-level phase) may be selected for motion correction, and the qualities of two corrected images are compared to evaluate a correction effect. (3) An optimal phase during systole or an optimal phase during diastole may be selected for reconstruction to observe a condition of myocardium or a coronary artery in different time phases, and the like. (4) A quality score of a to-be-evaluated image with an optimal phase without correction may be retrieved for comparison between different patients in combination with patient information (age, gender, heart rate, disease, and the like) to explore a correlation between different patient conditions and coronary artery qualities. In addition, it may also be used as a reference for quality control over scanning and cardiovascular imaging. The selection of phases is not limited in the present disclosure.

Based on the above, according to the method for determining a cardiac coronary artery imaging phase provided in the embodiments of the present disclosure, one or more target coronary arteries are extracted based on the plurality of phase images respectively to obtain a plurality of corresponding to-be-evaluated images, and weighted calculation is performed according to the image quality scores and the corresponding weighted parameters of the one or more target coronary arteries to obtain the quality score of each of the to-be-evaluated images, so that the user can customize the weighted parameters of different target coronary arteries as required, and an image quality score ranking corresponding to the global coronary artery qualities can be obtained after the quality score of each to-be-evaluated image is determined based on the weighted parameters of the one or more target coronary arteries. The required imaging phase of the cardiac coronary artery is determined based on the quality scores of the plurality of to-be-evaluated images, so that the user can determine an optimal phase for reconstruction according to a use purpose, thereby improving the imaging quality of the target coronary artery.

This embodiment of the present disclosure is described and illustrated below through preferred embodiments.

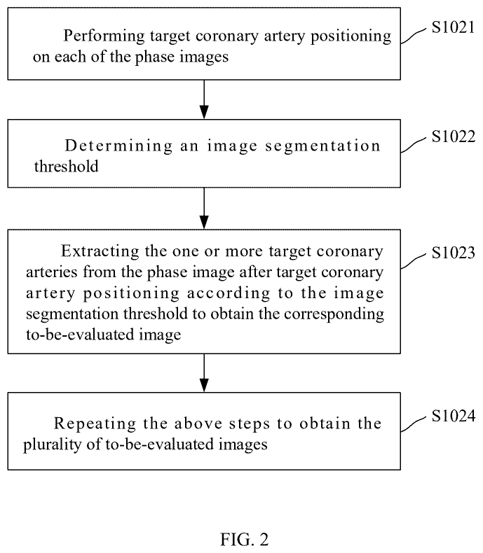

As shown in FIG. 2, on the basis of the above embodiments, in some embodiments, the extracting one or more target coronary arteries based on the plurality of phase images respectively to obtain a plurality of corresponding to-be-evaluated images includes the following steps S1021-S1024.

In step S1021, target coronary artery positioning is performed on each of the phase images.

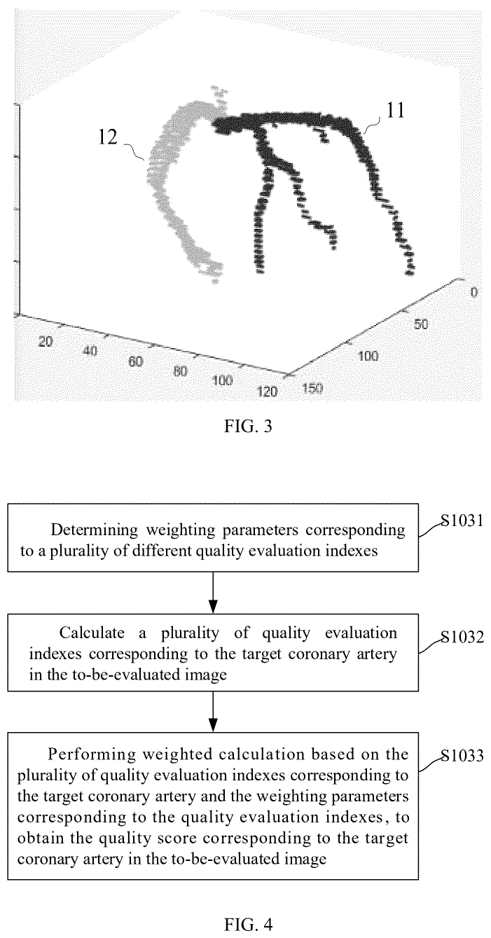

In this embodiment, the target coronary artery positioning may be performed by using a network model such as Vnet. Specifically, it is divided into four-layer up-sampling and four-layer down-sampling. Based on segmentation results, probability prediction is performed to determine whether a pixel in the phase image or a feature map is a foreground or background. A residual error is calculated by making a difference with a golden standard, and the residual error is optimized to train an entire model. The target coronary artery positioning is performed by using the trained model. It may be understood that the target coronary artery positioning may alternatively be performed by using another deep learning model. The model is not specifically limited in the present disclosure. Exemplarily, as shown in FIG. 3, the target coronary artery positioning is performed based on the Vnet network model to obtain a left coronary artery 11 and a right coronary artery 12.

In step S1022, an image segmentation threshold is determined.

In this embodiment, the image segmentation threshold may be a preset value or may be calculated and obtained, for example, equal to a preset multiple of a maximum value of the pixels in each phase image, or may be obtained by using an image processing method, or the like. The image segmentation threshold may be customized.

In some embodiments, the image segmentation threshold may be obtained by calculation. Determining an image segmentation threshold includes the following steps: acquiring a pixel value or CT value of the phase image; acquiring a preset subdivision parameter corresponding to the target coronary artery; and calculating the image segmentation threshold of the phase image according to the pixel value or CT value and the subdivision parameter. A calculation formula is as follows:

TL=T×Q

where TL denotes the image segmentation threshold, T denotes an image segmentation benchmark.

A pixel value of a phase image includes pixel values of all pixels of the phase image, and a CT value of the phase image includes CT values of all the pixels of the phase image. The pixel value may include a gray value. A preset subdivision parameter Q of the target coronary artery is a reference benchmark for a degree of image segmentation subdivision. The preset subdivision parameter Q may be configured as 0-1. Specifically, shapes of artifacts extracted by using different subdivision parameters Q are different. The lower the subdivision parameter Q is, the more motion artifacts are included in images obtained by segmentation. When an image subdivision program has a higher requirement, the preset subdivision parameter Q is larger. Otherwise, the preset subdivision parameter Q is smaller.

In some implementations, the image segmentation benchmark T may be determined according to the pixel value, and the image segmentation threshold TL of the phase image may be calculated according to the image segmentation benchmark T and the subdivision parameter Q. Optionally, the image segmentation benchmark T may be a maximum value in the pixel values of all the pixels of the phase image.

In some other implementations, the image segmentation benchmark T may be determined according to the CT value, and the image segmentation threshold TL of the phase image may be calculated according to the image segmentation benchmark T and the subdivision parameter Q. Optionally, the image segmentation benchmark T may be a median in the CT values of all the pixels of the phase image.

It may be understood that the manner of determining the image segmentation benchmark T is not limited thereto, and an Otsu method, a fixed parameter, or the like may also be obtained by using another image processing method. The image segmentation threshold may be adaptively configured.

It is to be noted that, prior to determining an image segmentation threshold, if image preprocessing (such as image enhancement) is performed on the phase image, the pixel value of the pixel in the phase image is different from the CT value of the pixel in the phase image. In this case, the image segmentation criterion T can be determined by using a pixel value of the preprocessed phase image or a CT value of an original phase image.

Through the above steps, the image segmentation threshold can be determined flexibly, and the to-be-evaluated image including the target coronary artery and a motion artifact thereof can be extracted, so that the image quality of the coronary artery can be more accurately evaluated on the coronary artery including artifacts.

In step S1023, the target coronary arteries are extracted from the phase image after the target coronary artery positioning according to the image segmentation threshold to obtain the corresponding to-be-evaluated image.

In step S1024, the above steps are repeated to obtain the plurality of to-be-evaluated images.

Specifically, in this embodiment, the step S1023 includes taking pixels whose gray values in the phase image are greater than the image segmentation threshold as pixels of the corresponding to-be-evaluated image. A plurality of image regions in the to-be-evaluated image may be obtained by segmenting the phase image by using a plurality of image segmentation thresholds.

Through the above steps, the target coronary artery is extracted from the phase image after the target coronary artery positioning based on the image segmentation threshold to obtain the corresponding to-be-evaluated image, and the to-be-evaluated image can be accurately determined according to the image segmentation threshold, so that determination of an optimal imaging phase based on the to-be-evaluated image after the extraction of the target coronary artery can be more accurate.

Based on the above embodiment, in some embodiments, prior to extracting the target coronary artery from the phase image after the target coronary artery positioning according to the image segmentation threshold to obtain the corresponding to-be-evaluated image, the method further includes at least one of the following processing steps.

In step S1022A, a segmentation center is marked according to a position of the target coronary artery in the phase image, and a segmentation region is delimited based on the segmentation center, so as to segment the phase image based on the segmentation region.

Specifically, sub-images may be obtained by pre-segmenting the phase image based on the segmentation center and the delimited segmentation region, and N*N pixels are taken as the sub-images after segmentation by taking the segmentation center as a center point. A calculation process is as follows:

I_{sub}=I(R1: R2,R3: R4) R2−R1=R4−R3=N−1

where I(R1: R2, R3: R4) denotes a pixel index range; R₁ and R₂ respectively denote a row starting point and a row ending point in the segmentation region; R₃ and R₄ respectively denote a column starting point and a column ending point in the segmentation region; N denotes a number of pixels of a row or column; I_{sub} denotes a pixel block size; and sub denotes a pixel index.

It may be understood that N may be customized and can cover a complete coronary artery in the phase image. The segmentation center may be determined by taking a single marked coronary artery as a reference, or taking a plurality of coronary arteries as a reference, which is not limited herein in the present disclosure.

Through the above step, the phase image is segmented based on the segmented region to obtain sub-images, and a target coronary artery is extracted from the sub-images to obtain a corresponding to-be-evaluated image, which can reduce a calculation amount during the extraction of the target coronary artery.

In step S1022B, the phase image is reconstructed by an image interpolation operation. Specifically, prior to determining an image segmentation threshold, interpolation reconstruction may be performed on the phase image.

Through the above step, the phase image is reconstructed by the image interpolation operation, which can improve image resolution, thereby improving the accuracy of calculating a shape of a coronary artery and an edge of the coronary artery. Certainly, in other embodiments, if the resolution of the phase image is sufficient, there is no need to reconstruct the phase image by the image interpolation operation.

In step S1022C, a morphological operation is performed on the phase image to weaken an image background.

Specifically, tophat transformation may be performed on the phase image.

It is to be noted that, prior to extracting the target coronary artery from the phase image after the target coronary artery positioning according to the image segmentation threshold to obtain the corresponding to-be-evaluated image, in some embodiments, the method further includes at least one of the above processing steps S1022A to S1022C. When multiple processing steps in S1022A to S1022C are adopted, a number and a sequence of the processing steps are not limited in the present disclosure. Through the above step, the image background is weakened, and a region where the target coronary artery is located is highlighted.

As shown in FIG. 4, on the basis of the above embodiment, in some embodiments, the calculating image quality scores corresponding to the target coronary artery in the to-be-evaluated images includes calculating the image quality score corresponding to the target coronary artery in a single to-be-evaluated image, which includes the following steps S1031-S1033.

In step S1031, weighting parameters corresponding to a plurality of different quality evaluation indexes are determined.

For different coronary arteries or different positions of the same coronary artery, shape information corresponding to the target coronary artery is not the same. For example, a front end and a back end of the target coronary artery are elongated, with small roundness and large sharpness, which are different from a direction of a middle end. The shape information of the target coronary artery may be determined by image segmentation to obtain a vascular area and a direction and a shape of the coronary artery in a 2D image or a topological structure in 3D, which is not limited herein in the present disclosure.

For target coronary arteries with different shape information, degrees of importance of different evaluation indexes thereof are different. For example, an elongated coronary artery (hereinafter referred to as a long coronary artery (Long CA)) is required to have smaller roundness and larger sharpness. Therefore, on a cross-section of the long coronary artery, a weight of regularity is lower than that of boundary sharpness. In this embodiment, when there are a plurality of evaluation indexes, weighting parameters of different evaluation indexes may be customized, so that an evaluation result can reflect degrees of importance of different evaluation indexes and the evaluation result is more accurate.

In step S1032, a plurality of quality evaluation indexes corresponding to the target coronary artery in the to-be-evaluated image are calculated.

There is a clinical evaluation criterion in the evaluation of cardiovascular images: an ideal coronary artery should have clear edges, the second is that the edges are slightly blurred without obvious artifacts, and the passing is that an outline of the coronary artery is visible and some slight artifacts are allowed. Blurred coronary artery boundaries, severe motion artifacts, and disappearing coronary artery outlines are undiagnosable. A plurality of evaluation indexes may be proposed in combination with the above clinical evaluation criterion.

In this embodiment, the quality evaluation indexes of the target coronary artery may be shape regularity, boundary sharpness, and the like. The above two quality evaluation indexes are respectively used to measure whether a region of interest boundary is blurred and the strength of a motion artifact. The two criteria are respectively quantified as shape regularity (strength of the artifact) and boundary sharpness (a degree of sharpness of the boundary). The shape regularity and the boundary sharpness may basically cover coronary artery images with various levels of quality (including coronary arteries with stents, calcified coronary arteries, and the like), and are more versatile. Certainly, in other embodiments, shape features of the cardiac coronary artery may alternatively be quantified through other evaluation indexes, such as proportions of low CT values in cardiac coronary arteries (CT values of artifacts are generally lower than CT values of vascular contrast agents) or entropy. No limitation is made herein in the present disclosure.

In step S1033, weighted calculation is performed based on the plurality of quality evaluation indexes corresponding to the target coronary artery and the weighting parameters corresponding to the quality evaluation indexes, to obtain the quality score corresponding to the target coronary artery in the to-be-evaluated image.

Specifically, after the weighting parameters corresponding to the quality evaluation indexes are acquired, a value of the quality evaluation index and the corresponding weighting parameter of the target coronary artery are weighted and summed to obtain the image quality score corresponding to the target coronary artery in each to-be-evaluated image.

Exemplarily, the image quality score of the target coronary artery may be calculated by using the following formula:

$$QuaIdx = \{ \begin{matrix}regularity + sharpness \times factorS & if LongCA is false \\ regularity + sharpness \times factorSL & if LongCA is true\end{matrix}$$

where regularity denotes shape regularity; sharpness denotes boundary sharpness; factorS denotes a weighting parameter corresponding to the shape regularity; factorSL denotes a weighting parameter corresponding to the boundary sharpness; QuaIdx denotes the image quality score of the target coronary artery; if LongCA is false denotes when the target coronary artery is not a long coronary artery; if LongCA is true denotes when the target coronary artery is a long coronary artery; + denotes an addition operation; and × denotes a multiplication operation.

In addition, since magnitudes of the shape regularity and the boundary sharpness are inconsistent, there is a need to pull these two metrics to a baseline, either by weighting or by normalization, which is not limited herein in the present

### DISCLOSURE

Through the above step, weighting parameters corresponding to different quality evaluation indexes are determined. Therefore, weighting parameters of different evaluation indexes are customized, so that an evaluation result can reflect degrees of importance of different evaluation indexes and the evaluation result of the image quality score of the cardiac coronary artery is more accurate and reliable.

On the basis of the above embodiments, in some embodiments, determining a required imaging phase of the cardiac coronary artery based on the quality scores of the plurality of to-be-evaluated images includes the following steps S1061-S1063.

In step S1061, a plurality of to-be-evaluated images within a preset single phase sliding window are acquired, and filtering is performed to obtain common slices and extra slices that correspond to each time phase.

Time phases collected during the scanning of the cardiac coronary artery include a plurality of cardiac cycles. For example, in retrospective cardiac scanning, the collected time phases cover an entire cardiac cycle (0% to 100%). In prospective scanning, the cardiac cycles generally may include systole and diastole, and motions of the coronary arteries in a direction z are inconsistent in different cardiac cycles such as systole and diastole. In a real clinical scenario, evaluation of the to-be-evaluated image based on a 2D cross section is required to consider inconsistency of the coronary arteries in the direction z under different phases. Therefore, the calculation of the image quality score in a single to-be-evaluated image cannot be simply compared under the same z-direction coordinates. The direction z refers to a body length direction.

In this embodiment, the phase sliding window refers to a phase range within a preset window width in the entire cardiac cycle. In a single phase sliding window, motion patterns of the coronary arteries in the direction z are considered to be similar, and a time length of the phase sliding window may be adaptively configured. Exemplarily, the length of the phase sliding window may be adaptively adjusted according to a heart rate. The length of the phase sliding window should not be too wide. Optionally, the phase sliding window ranges from 10% to 20% of an entire cardiac cycle. A preset single phase sliding window includes multiple time phases

In this embodiment, the common slices and the extra slices corresponding to each time phase can be obtained by filtering based on a plurality of to-be-evaluated images within a to-be-evaluated image phase sliding window. The common slices corresponding to each time phase are to-be-evaluated images with same image reconstruction layers in each time phase. The extra slices corresponding to each time phase is other to-be-evaluated images except the common slices.

In step S1062, based on image quality scores of the common slices and the extra slices in a single time phase and a number of image layers of the single time phase, an image quality score of the corresponding time phase is calculated.

In this embodiment, firstly, based on quality scores of the common slices and a number of layers of each time phase with the common slices in the single time phase within the single phase sliding window, an average score of the corresponding time phase is calculated. Specifically, a quality score mean value VQSAvg is determined based on the image quality score of each time phase with the common slices in the single time phase within the single phase sliding window. A layer number mean value AvgRange is determined based on the number of layers of each time phase with the common slices in the single time phase within the single phase sliding window. A product of the quality score mean value VQSAvg and the layer number mean value AvgRange is determined as the average score of the corresponding time phase.

Then, an inter-phase offset score VQOffset is calculated according to a number of layers of the common slices in the single time phase and a mean value of a number of layers of each time phase. Specifically, a difference between the number of layers of the common slices in the single time phase and the mean value of the number of layers of each time phase may be determined as the inter-phase offset score VQOffset. It may be understood that the inter-phase offset score may be used to represent a phase offset of a single phase, and a calculation manner thereof is not limited thereto.

Next, an intra-phase offset score VQExt is determined based on quality scores of the extra slices in the single phase. In some embodiments, the intra-phase offset score VQExt may be the quality scores of the extra slices. In some other embodiments, the intra-phase offset score VQExt may be a difference between the quality scores of the extra slices and the quality score mean value VQSAvg, which is not limited herein in the present disclosure.

Finally, weighted calculation is performed according to the average score, the inter-phase offset score VQOffset, and the intra-phase offset score VQExt to obtain the image quality score VQ of the corresponding time phase.

VQ=VQSAvg×AvgRange+VQOffset×w1+VQExt×w2

where w1 and w2 are weighting coefficients, ranging from 0 to 1.

It may be understood that, in other embodiments, the manner of calculating the image quality score of the corresponding time phase is not limited thereto. For example, the image quality scores of the common slices and the extra slices in a single time phase and the number of image layers of the single time phase are acquired and then may be directly weighted and summed, and normalized to obtain the image quality score of the corresponding phase, which is not limited herein in the present disclosure.

In step S1063, the above steps are repeated to calculate quality scores of a plurality of to-be-evaluated images in each time phase within the single phase sliding window, and the required imaging phase of the cardiac coronary artery is determined according to the image quality scores.

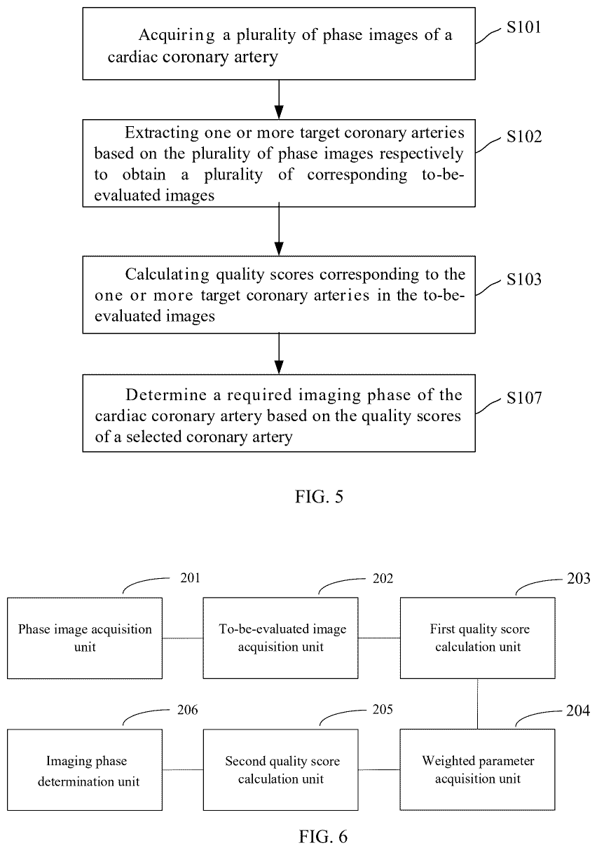

FIG. 5 schematic flowchart of a method for determining a cardiac coronary artery imaging phase according to another embodiment of the present disclosure. The steps S101-S103 are the same as the corresponding steps in the corresponding embodiment of FIG. 1 and are not repeated herein respectively. In this embodiment, the method further includes step S107.

In the step S107, a required imaging phase of the cardiac coronary artery is determined based on the quality scores of a selected coronary artery.

In some embodiments, a specific coronary artery is selected as the selected coronary artery. An optimal phase is determined based on the quality scores of the selected coronary artery and considered as the required imaging phase. For example, the left coronary artery may be selected as the selected coronary artery, and the quality score of the left coronary artery in each of the to-be-evaluated images is calculated. Based on the quality scores of the left coronary artery, a phase ranking is performed and the optimal phase is selected as the required imaging phase of the cardiac coronary artery.

In this embodiment, determining the required imaging phase directly based on the selected coronary artery avoids the weighted calculation on multiple different target coronary arteries, thus further enhancing the efficiency of determining the required phase.

In this embodiment, the above steps are repeated to calculate quality scores of a plurality of to-be-evaluated images in each time phase within the single phase sliding window, the quality scores of the plurality of to-be-evaluated images in each time phase are sorted, and phases at different ranking positions are selected for reconstruction according to the use purpose of the phase image. Through the above steps, a phase sliding window is introduced to calculate the quality scores of the plurality of to-be-evaluated images in each time phase, motion patterns of the coronary arteries in the direction z are considered to be similar in a single phase sliding window, which can better match the variability of the coronary arteries in the direction z in different time phases, making a calculation result of the image quality score more accurate, thereby ensuring the reliability of an optimal imaging phase and imaging quality of the target coronary artery.

In some embodiments, image registration in the z-direction is performed on the target coronary artery of the to-be-evaluated image in each time phase, so that the position of the target coronary artery in the z-direction corresponding to each phase is confirmed. The required imaging phase can be determined by selecting the quality scores of the to-be-evaluated images within a preset similarity range for comparison and analysis based the positions of the target coronary artery after image registration, and the accuracy of the determination is thus improved.

It is to be noted that the steps shown in the above flowcharts or in the flowcharts of the accompanying drawings may be implemented in a computer system of such as a set of computer-executable instructions. Moreover, although a logical order is shown in the flowcharts, in some cases, the steps shown or described may be performed in an order different from that shown or described herein.

This embodiment further provides a cardiac coronary artery imaging phase determination apparatus. The apparatus is configured to implement the above embodiment and preferred implementations, and those that have been described may not be described in detail. As used below, the terms “module”, “unit”, “subunit”, and the like may be a combination of software and/or hardware that implement a predetermined function. Although the apparatus described in the following embodiment is preferably implemented with software, implementations by hardware, or a combination of software and hardware are also possible and conceived.

FIG. 6 is a structural block diagram of a cardiac coronary artery imaging phase determination apparatus according to an embodiment of the present disclosure. As shown in FIG. 6, the apparatus includes a phase image acquisition unit 201, a to-be-evaluated image acquisition unit 202, a first quality score calculation unit 203, a weighted parameter acquisition unit 204, a second quality score calculation unit 205, and an imaging phase determination unit 206.

The phase image acquisition unit 201 is configured to acquire a plurality of phase images of a cardiac coronary artery.

The to-be-evaluated image acquisition unit 202 is configured to extract one or more target coronary arteries based on the plurality of phase images respectively to obtain a plurality of corresponding to-be-evaluated images.

The first quality score calculation unit 203 is configured to calculate image quality scores corresponding to the one or more target coronary arteries in the to-be-evaluated images.

The weighted parameter acquisition unit 204 is configured to, when at least two target coronary arteries are included, acquire weighted parameters corresponding to the target coronary arteries.

The second quality score calculation unit 205 is configured to perform a weighted calculation according to the image quality scores and the corresponding weighted parameters of the one or more target coronary arteries to obtain the quality score of each of the to-be-evaluated images.

The imaging phase determination unit 206 is configured to determine a required imaging phase of the cardiac coronary artery based on the quality scores of the plurality of to-be-evaluated images.

In some embodiments, the to-be-evaluated image acquisition unit 202 includes: a target coronary artery positioning module, a threshold determination module, a target coronary artery extraction module, and a circulation module.

The target coronary artery positioning module is configured to perform target coronary artery positioning on the phase image.

The threshold determination module is configured to determine an image segmentation threshold.

The target coronary artery extraction module is configured to extract the target coronary artery from the phase image after the target coronary artery positioning according to the image segmentation threshold to obtain the corresponding to-be-evaluated image.

The circulation module is configured to repeat the above steps to obtain the plurality of to-be-evaluated images.

In some embodiments, the threshold determination module includes a pixel parameter acquisition module, a subdivision parameter acquisition module, and a segmentation threshold calculation module.

The pixel parameter acquisition module is configured to acquire a pixel value or CT value of the phase image.

The subdivision parameter acquisition module is configured to acquire a preset subdivision parameter corresponding to the target coronary artery.

The segmentation threshold calculation module is configured to calculate the image segmentation threshold of the phase image according to the pixel value or CT value and the subdivision parameter.

In some embodiments, the cardiac coronary artery imaging phase determination apparatus further includes at least one of the following: a first preprocessing module, a second preprocessing module, and a third preprocessing module.

The first preprocessing module is configured to mark a segmentation center according to a position of the target coronary artery in the phase image, and delimit a segmentation region based on the segmentation center, so as to segment the phase image based on the segmentation region.

The second preprocessing module is configured to reconstruct the phase image by an image interpolation operation.

The third preprocessing module is configured to perform a morphological operation on the phase image to weaken an image background.

In some embodiments, the first quality score calculation unit 203 includes a weighting parameter determination module, an evaluation index calculation module, and a first quality score calculation module.

The weighting parameter determination module is configured to determine weighting parameters corresponding to a plurality of different quality evaluation indexes.

The evaluation index calculation module is configured to calculate a plurality of quality evaluation indexes corresponding to the target coronary artery in the to-be-evaluated image.

The first quality score calculation module is configured to perform a weighted calculation based on the plurality of quality evaluation indexes corresponding to the target coronary artery and the weighting parameters corresponding to the quality evaluation indexes, to obtain the quality score corresponding to the target coronary artery in the to-be-evaluated image.

In some embodiments, the imaging phase determination unit 206 includes an image acquisition module configured to acquire a plurality of to-be-evaluated images within a preset single phase sliding window and perform filtering to obtain common slices and extra slices that correspond to each time phase, an image quality score calculation module configured to calculate, based on image quality scores of the common slices and the extra slices in a single time phase and a number of image layers of the single time phase, an image quality score of the corresponding time phase, and a phase determination module configured to repeat the above steps to calculate quality scores of a plurality of to-be-evaluated images in each time phase within the single phase sliding window and determine the imaging phase of the cardiac coronary artery according to the image quality scores.

In some embodiments, the image quality score calculation module includes an average score calculation module configured to calculate, based on quality scores of the common slices and a number of layers of each time phase with the common slices in the single time phase within the single phase sliding window, an average score of the corresponding time phase, an inter-phase offset score calculation module configured to calculate an inter-phase offset score according to a number of layers of the common slices in the single time phase and a mean value of a number of layers of each time phase, an intra-phase offset score calculation module configured to determine an intra-phase offset score based on quality scores of the extra slices in the single phase, and a weighted calculation module configured to perform a weighted calculation according to the average score, the inter-phase offset score, and the intra-phase offset score to obtain the image quality score of the corresponding time phase.

It should be noted that each of the above modules may be a function module or a program module, and may be implemented by software or hardware. For the modules implemented by hardware, the above modules may be located in a same processor; or the above modules may alternatively be located in different processors in any combination.

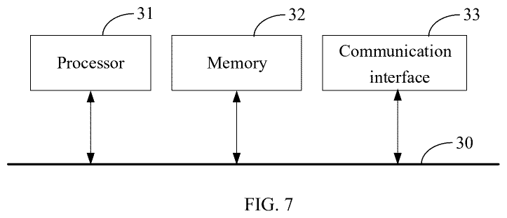

In addition, a method for determining a cardiac coronary artery imaging phase according to an embodiment of the present disclosure described with reference to FIG. 7 may be implemented by an electronic device. FIG. 7 is a schematic diagram of a hardware structure of an electronic device according to an embodiment of the present disclosure.

The electronic device may include a processor 31 or a memory 32 storing computer program instructions.

Specifically, the above processor 31 may include a central processing unit (CPU), or an Application Specific Integrated Circuit (ASIC), or may be configured as one or more integrated circuits implementing this embodiment of the present disclosure.

The memory 32 may include a mass memory for data or instructions. As an example and not by way of limitation, the memory 32 may include a Hard Disk Drive (HDD), a floppy disk drive, a Solid State Drive (SSD), a flash memory, an optical disk, a magneto-optical disk, a magnetic tape or a Universal Serial Bus (USB) drive, or a combination of two or more thereof. The memory 32 may include a removable or non-removable (or fixed) medium, where appropriate. The memory 32 may be inside or outside a data processing apparatus, where appropriate. In a particular embodiment, the memory 32 is a non-volatile memory. In a particular embodiment, the memory 32 includes a Read-Only Memory (ROM) and a Random Access Memory (RAM). Where appropriate, the ROM may be a mask-programmed ROM, a Programmable Read-Only Memory (PROM), an Erasable Programmable Read-Only Memory (EPROM), an Electrically Erasable Programmable Read-Only Memory (EEPROM), an Electrically Alterable Read-Only Memory (EAROM), a flash, or a combination of two or more thereof. Where appropriate, the RAM may be a Static Random-Access Memory (SRAM) or a Dynamic Random Access Memory (DRAM). The DRAM may be a Fast Page Mode Dynamic Random Access Memory (FPMDRAM), an Extended Date Out Dynamic Random Access Memory (EDODRAM), a Synchronous Dynamic Random-Access Memory (SDRAM), or the like.

The memory 32 may be configured to store or cache various data files required for processing and/or communication, as well as possible computer program instructions executed by the processor 31.

The processor 31 reads and executes the computer program instructions stored in the memory 32 to implement the method for determining a cardiac coronary artery imaging phase in any one of the above embodiments.

In some embodiments, the electronic device may further include a communication interface 33 and a bus 30. As shown in FIG. 7, the processor 31, the memory 32, and the communication interface 33 are connected through the bus 30 and complete mutual communication.

The communication interface 33 is configured to implement communication between various modules, apparatuses, units, and/or devices in this embodiment of the present disclosure. The communication interface 33 may further implement data communication with other components such as an external device, an image/data collection device, a database, an external storage, and an image/data processing workstation.

The bus 30 includes hardware, software, or both, and couples components of the electronic device to each other. The bus 30 includes, but is not limited to, at least one of the following: a data bus, an address bus, a control bus, an expansion bus, and a local bus. As an example and not by way of limitation, the bus 30 may include an Accelerated Graphics Port (AGP) or other graphics bus, an Enhanced Industry Standard Architecture (EISA) bus, a Front Side Bus (FSB), a Hyper Transport (HT) interconnect, an Industry Standard Architecture (ISA) bus, an Infiniband interconnect, a Low Pin Count (LPC) bus, a memory bus, a Micro Channel Architecture (MCA) bus, a Peripheral Component Interconnect (PCI) bus, a PCI-Express (PCI-X) bus, a Serial Advanced Technology Attachment (SATA) bus, a Video Electronics Standards Association local (VLB) bus, or another suitable bus or a combination of two or more thereof. The bus 30 may include one or more buses, where appropriate. Although this embodiment of the present disclosure describes and illustrates a particular bus, the present disclosure contemplates any suitable bus or interconnect.

The electronic device may perform the method for determining a cardiac coronary artery imaging phase in the embodiments of the present disclosure based on an acquired program instruction, so as to implement the method for determining a cardiac coronary artery imaging phase described with reference to FIG. 1.

In addition, in combination with the method for determining a cardiac coronary artery imaging phase in the above embodiment, an embodiment of the present disclosure may provide a computer-readable storage medium for implementation. The computer-readable storage medium stores a computer program instruction. When the computer program instruction is executed by a processor, the method for determining a cardiac coronary artery imaging phase in any one of the above embodiments is implemented.

The technical features in the above embodiments may be randomly combined. For concise description, not all possible combinations of the technical features in the above embodiments are described. However, all the combinations of the technical features are to be considered as falling within the scope described in this specification provided that they do not conflict with each other.

The above embodiments only describe several implementations of the present disclosure, and their description is specific and detailed, but cannot therefore be understood as a limitation on the patent scope of the invention. It should be noted that those of ordinary skill in the art may further make variations and improvements without departing from the conception of the present disclosure, and these all fall within the protection scope of the present disclosure. Therefore, the patent protection scope of the present disclosure should be subject to the appended claims.

What is claimed is:

1. A method for determining a cardiac coronary artery imaging phase, comprising:
acquiring a plurality of phase images of a cardiac coronary artery;
extracting one or more target coronary arteries based on the plurality of phase images respectively to obtain a plurality of corresponding to-be-evaluated images;
calculating image quality scores of the one or more target coronary arteries in each of the to-be-evaluated images;
performing a weighted calculation according to the image quality scores of the one or more target coronary arteries and weighted parameters of the one or more target coronary arteries to obtain a quality score of each of the to-be-evaluated images; and
determining a required imaging phase of the cardiac coronary artery based on the quality scores of the plurality of to-be-evaluated images.

2. The method according to claim 1, wherein the extracting one or more target coronary arteries based on the plurality of phase images respectively to obtain the plurality of corresponding to-be-evaluated images comprises:
performing target coronary artery positioning on each of the phase images;
determining an image segmentation threshold; and
extracting the one or more target coronary arteries from the phase image after the target coronary artery positioning according to the image segmentation threshold to obtain the corresponding to-be-evaluated image.

3. The method according to claim 2, wherein the determining the image segmentation threshold comprises:
acquiring a pixel value or CT value of the phase image;
acquiring a preset subdivision parameter corresponding to the target coronary artery; and
calculating the image segmentation threshold of the phase image according to the pixel value or CT value and the subdivision parameter.

4. The method according to claim 2, prior to the extracting the one or more target coronary arteries from the phase image after the target coronary artery positioning according to the image segmentation threshold to obtain the corresponding to-be-evaluated image, further comprising:
marking a segmentation center according to a position of the target coronary artery in the phase image, and delimiting a segmentation region based on the segmentation center, so as to segment the phase image based on the segmentation region.

5. The method according to claim 2, prior to the extracting the one or more target coronary arteries from the phase image after the target coronary artery positioning according to the image segmentation threshold to obtain the corresponding to-be-evaluated image, further comprising:
reconstructing the phase image by an image interpolation operation.

6. The method according to claim 2, prior to the extracting the one or more target coronary arteries from the phase image after the target coronary artery positioning according to the image segmentation threshold to obtain the corresponding to-be-evaluated image, further comprising:
performing a morphological operation on the phase image to weaken an image background.

7. The method according to claim 1, wherein the calculating image quality scores of the one or more target coronary arteries in each of the to-be-evaluated images comprises:
calculating a plurality of quality evaluation indexes corresponding to the one or more target coronary arteries in each of the to-be-evaluated images;
determining weighting parameters of the plurality of quality evaluation indexes; and
performing a weighted calculation based on the plurality of quality evaluation indexes corresponding to the one or more target coronary arteries and the weighting parameters of the quality evaluation indexes, to obtain the quality scores of the one or more target coronary arteries in the to-be-evaluated image.

8. The method according to claim 1, wherein the determining the required imaging phase of the cardiac coronary artery based on the quality scores of the plurality of to-be-evaluated images comprises:
acquiring the plurality of to-be-evaluated images within a preset single phase sliding window including multiple time phases, and performing filtering to obtain common slices and extra slices that correspond to each time phase;
calculating, based on image quality scores of the common slices and the extra slices in a single time phase and a number of image layers of the single time phase, an image quality score of the corresponding time phase; and
repeating the above steps to calculate the quality scores of the plurality of to-be-evaluated images in each time phase within the single phase sliding window, and determining the required imaging phase of the cardiac coronary artery according to the quality scores the plurality of to-be-evaluated images.

9. The method according to claim 8, wherein the calculating, based on the image quality scores of the common slices and the extra slices in the single time phase and the number of image layers of the single time phase, the image quality score of the corresponding time phase comprises:
calculating, based on quality scores of the common slices and a number of layers of each time phase with the common slices in the single time phase within the single phase sliding window, an average score of the corresponding time phase;
calculating an inter-phase offset score according to a number of layers of the common slices in the single time phase and a mean value of a number of layers of each time phase;
determining an intra-phase offset score based on quality scores of the extra slices in the single phase; and
performing a weighted calculation according to the average score, the inter-phase offset score, and the intra-phase offset score to obtain the image quality score of the corresponding time phase.

10. The method according to claim 8, further comprising:
performing image registration in the z-direction on the at least one target coronary artery of the to-be-evaluated image in each time phase to determine a position of the at least one target coronary artery in the z-direction corresponding to each time phase;
selecting the to-be-evaluated images within a preset similarity range based the positions of the at least one target coronary artery after image registration; and
determining the required imaging phase of the cardiac coronary artery based on quality scores of the selected to-be-evaluated images.

11. An electronic device, comprising a memory and a processor, wherein the memory stores a computer program, and the processor is configured to execute the computer program to perform a method for determining a cardiac coronary artery imaging phase which includes:
acquiring a plurality of phase images of a cardiac coronary artery;
extracting one or more target coronary arteries based on the plurality of phase images respectively to obtain a plurality of corresponding to-be-evaluated images;
calculating image quality scores of the one or more target coronary arteries in each of the to-be-evaluated images;
performing a weighted calculation according to the image quality scores of the one or more target coronary arteries and the weighted parameters of the one or more target coronary arteries to obtain a quality score of each of the to-be-evaluated images; and
determining a required imaging phase of the cardiac coronary artery based on the quality scores of the plurality of to-be-evaluated images.

12. The electronic device according to claim 11, wherein the extracting one or more target coronary arteries based on the plurality of phase images respectively to obtain the plurality of corresponding to-be-evaluated images comprises:
performing target coronary artery positioning on each of the phase images;
determining an image segmentation threshold; and
extracting the one or more target coronary arteries from the phase image after the target coronary artery positioning according to the image segmentation threshold to obtain the corresponding to-be-evaluated image.

13. The electronic device according to claim 12, wherein the determining the image segmentation threshold comprises:
acquiring a pixel value or CT value of the phase image;
acquiring a preset subdivision parameter corresponding to the target coronary artery; and
calculating the image segmentation threshold of the phase image according to the pixel value or CT value and the subdivision parameter.

14. The electronic device according to claim 12, prior to the extracting the one or more target coronary arteries from the phase image after the target coronary artery positioning according to the image segmentation threshold to obtain the corresponding to-be-evaluated image, further comprising:
marking a segmentation center according to a position of the target coronary artery in the phase image, and delimiting a segmentation region based on the segmentation center, so as to segment the phase image based on the segmentation region.

15. The electronic device according to claim 12, prior to the extracting the one or more target coronary arteries from the phase image after the target coronary artery positioning according to the image segmentation threshold to obtain the corresponding to-be-evaluated image, further comprising:
reconstructing the phase image by an image interpolation operation.

16. The electronic device according to claim 12, prior to the extracting the one or more target coronary arteries from the phase image after the target coronary artery positioning according to the image segmentation threshold to obtain the corresponding to-be-evaluated image, further comprising:
performing a morphological operation on the phase image to weaken an image background.

17. The electronic device according to claim 11, wherein the calculating image quality scores of the one or more target coronary arteries in each of the to-be-evaluated images comprises:
calculating a plurality of quality evaluation indexes corresponding to the one or more target coronary arteries in each of the to-be-evaluated images;
determining weighting parameters of the plurality of quality evaluation indexes; and
performing a weighted calculation based on the plurality of quality evaluation indexes corresponding to the one or more target coronary arteries and the weighting parameters of the quality evaluation indexes, to obtain the quality scores of the one or more target coronary arteries in the to-be-evaluated image.

18. The electronic device according to claim 11, wherein the determining the required imaging phase of the cardiac coronary artery based on the quality scores of the plurality of to-be-evaluated images comprises:
acquiring the plurality of to-be-evaluated images within a preset single phase sliding window including multiple time phases, and performing filtering to obtain common slices and extra slices that correspond to each time phase;
calculating, based on image quality scores of the common slices and the extra slices in a single time phase and a number of image layers of the single time phase, an image quality score of the corresponding time phase; and
repeating the above step to calculate the quality scores of the plurality of to-be-evaluated images in each time phase within the single phase sliding window, and determining the required imaging phase of the cardiac coronary artery according to the quality scores the plurality of to-be-evaluated images.

19. The electronic device according to claim 18, wherein the calculating, based on the image quality scores of the common slices and the extra slices in the single time phase and the number of image layers of the single time phase, the image quality score of the corresponding time phase comprises:
calculating, based on quality scores of the common slices and a number of layers of each time phase with the common slices in the single time phase within the single phase sliding window, an average score of the corresponding time phase;
calculating an inter-phase offset score according to a number of layers of the common slices in the single time phase and a mean value of a number of layers of each time phase;
determining an intra-phase offset score based on quality scores of the extra slices in the single phase; and
performing a weighted calculation according to the average score, the inter-phase offset score, and the intra-phase offset score to obtain the image quality score of the corresponding time phase.

20. A computer-readable storage medium, storing a computer program, wherein the computer program, when executed by a processor, causes the processor to perform method for determining a cardiac coronary artery imaging phase which includes:
acquiring a plurality of phase images of a cardiac coronary artery;
extracting one or more target coronary arteries based on the plurality of phase images respectively to obtain a plurality of corresponding to-be-evaluated images;
calculating image quality scores of the one or more target coronary arteries in each of the to-be-evaluated images;
performing a weighted calculation according to the image quality scores of the one or more target coronary arteries and the weighted parameters of the one or more target coronary arteries to obtain a quality score of each of the to-be-evaluated images; and
determining a required imaging phase of the cardiac coronary artery based on the quality scores of the plurality of to-be-evaluated images.
